(19) United States
(12) Patent Application Publication
(10) Pub. No.: US 2018/0318206 A1
(43) Pub. Date: Nov. 8, 2018

(54) A PRODUCT PREVENTING HAIR LOSS

(71) Applicant: YEDITEPE UNIVERSITESI, Istanbul (TR)

(72) Inventors: Fikrettin SAHIN, Istanbul (TR); Ozge Sezin SOMUNCU, Istanbul (TR); Pakize Neslihan TASLI, Istanbul (TR); Okan DEMIR, Istanbul (TR)

(73) Assignee: YEDITEPE UNIVERSITESI, Istanbul (TR)

(21) Appl. No.: 15/772,864

(22) PCT Filed: Nov. 1, 2016
(86) PCT No.: PCT/TR2016/050412

§ 371 Date: May 2, 2018

(30) Foreign Application Priority Data
Nov. 2, 2015 (TR) 2015/13573

(51) Int. Cl. A61K 8/98; A61Q 7/00

(52) U.S. Cl. CPC A61K 8/985; A61Q 7/00

(57) ABSTRACT

The present invention relates to a product which prevents hair loss and enables growth of healthy new hair by stem cell application. With the present invention, the newly growing hair is ensured to have the same color with the person's own hair color and thus prevents increase of grey hair. The product of the present invention is comprised of a carrier and active ingredient.

### CROSS REFERENCE TO THE RELATED APPLICATIONS

This application is the national phase entry of International Application No. PCT/TR2016/050412, filed on Nov 1, 2016, which claims priority from Turkish Patent Application 2015/13573, filed on Nov 2, 2015, the entire contents of which are incorporated herein by reference.

### TECHNICAL FIELD

The present invention relates to a product which prevents hair loss and enables growth of healthy new hair by application of the exosome released by the stem cell to the medium.

### BACKGROUND

Intense hair loss is a problem which can be encountered both in men and women. There are many reasons of this problem depending on hormones, age, stress and mineral deficiency.

Mammalian cells include small vesicular structures called exosome. When these exosomes are isolated from healthy cells, they can be used to restore the cells, which are damaged or under a certain stress and which cannot complete their self-regeneration, back to their earlier healthy state. Recently, use of the stem cells in cell based therapies is also one of the most promising studies. It is asserted that use of the exosomes released from these cells will have positive effects on wound healing and hair growth/strengthening in clinical application.

It is seen in the studies that human newborn foreskin stem cells have a much better regeneration ability compared to the other stem cells, and they are a good source since they are waste tissues. Viability abilities and reprogramming abilities of these stem cells are much better than many of the stem cells.

United States patent document no. US20130209528 discloses a pharmaceutical composition which enhances hair growth by use of exosomes which are derived from a mesenchymal stem cell.

### SUMMARY

The objective of the present invention is to provide a product which prevents hair loss.

Another objective of the present invention is to provide a product which enables hair with weak bulbs to get stronger and more abundant. A further objective of the present invention is to provide a product which enables the newly growing hair to have the same color with the person's own hair color and thus prevents increase of grey hair.

### BRIEF DESCRIPTION OF THE DRAWINGS

“A product preventing hair loss” is supported by the accompanying figures, wherein

FIG. 1 is the view of the characterization obtained by the flow cytometer via the stem cell surface markers of the human newborn foreskin stem cells.

FIG. 2 is the
c—electron microscope image at 7.00 KX
d—electron microscope image at 15.00 KX. of human newborn foreskin stem cell exosomes.

FIG. 3 is the view of
c—the thickness of the hair of the mouse on which exosome application is not performed and
d—the thickness of the hair isolated from the mouse on which exosome application is performed.

FIG. 4 is the view of
c—the hair thickness and scarcity of the mouse skin on which exosome application is not performed
d—the hair thickness and abundance on which exosome application is performed.

### DETAILED DESCRIPTION

In the embodiment of the invention, human newborn foreskin stem cells were used. It has been determined that differentiation potential of these cells is different from that of the other stem cells. However, the mechanisms that control the differentiation ability of these cells are not known. In the present study, it is determined that the exosomes carried by these cells have an inductive molecular content in cell differentiation. It has been determined that the said exosomes cause hair growth by inducing the hair cells in topical and subcutaneous applications. Therefore, various formulations are developed with this molecular mixture containing exosome and they are used for baldness treatment.

### Experimental Studies

### Stem Cell Isolation From Newborn Foreskin

Stem cells were isolated from foreskins of 0 to 5 month old babies by known methods. These stem cells were grown in Dulbecco's modified essential medium (DMEM) which contained 10% fetal bovine serum and 1% PSA (penicillin, streptomycin and amphotericin), in an incubator at a temperature of 37° C. and in 5% CO₂ atmosphere.

### Characterization of Stem Cells

After the cells were trypsinized in the culture medium, they were treated for 1 hour with primary antibodies diluted in PBS (PBS; cat #10010, pH 7.4; Invitrogen). CD29 (cat #BD556049), CD34 (cat #SC-51540), CD45 (cat #SC-70686), CD90 (cat #SC-53456), CD105 (cat #SC-71043), CD31 (cat #SC-65278), CD166 (cat #SC-53551) (Santa Cruz Biotechnology Inc, Santa Cruz, Calif.), and CD73 (cat #D 550256) (Zymed, San Francisco, Calif.) primary antibodies were used for characterization. The cells were washed with PBS three times for removing the excess unbonded primary antibodies in the medium. Flow cytometry analysis was performed by using Becton Dickinson FACSCalibur (Becton Dickinson, San Jose, Calif.) system.

### Exosome Isolation From Cultured Stem Cells

EX01 Exo-spin™ kit was used for exosome isolation from cultured foreskin stem cells. The medium of the cells, which occupy 80% of the culture medium, was collected, and it was centrifuged at 300× g for 10 minutes in order to remove the waste cells. The supernatant was transferred to a new tube and it was centrifuged at 16000× g for 30 minutes in order to remove possible cell components. The supernatant was transferred to a new tube and ½ volume of solution A was added, and it was allowed to rest for one day at +4 degrees. The next day, it was centrifuged at 16000× g for 1 hour and the pellet was dissolved in PBS.

### Cytotoxicity Experiment

6 different concentrations of the isolated exosomes (5-3-1-0.5-0.25-0.1 μg/ml) were prepared in Dulbecco's modified Eagle's medium (DMEM) containing 10% fetal bovine serum (Invitrogen) and 1% PSA (Biological Industries, Beit Haemek, Israel). 24 hours after being seeded in 96-well culture plates (Corning Glasswork, Corning, N.Y.) at 5000 cells/well, the HACAT cells were treated for 3 days with exosomes prepared at different concentrations. Cell viability was measured by using 3-(4,5-di-methyl-thiazol-2-yl)-5-(3-carboxy-methoxy-phenyl)-2-(4-sulfo-phenyl)-2H-tetrazolium (MTS)-method (CellTiter96 AqueousOne Solution; Promega, Southampton, UK). 10 μl MTS solution was added onto the cells within a 100 μl growth medium and they were incubated in dark for 2 hours. After the incubation process, cell viability was observed by performing absorbance measurement via ELISA plate reader (Biotek, Winooski, Vt.) device at 490 nm wavelength.

### Preparing the Product Content

The product, which prevents hair loss and enables the newly growing hair to be natural and healthy, has the following content;
The product of the present invention is comprised of a carrier+active ingredient.
The carrier may comprise natural and chemical oils, synthetic and semisynthetic polymers, biopolymers, hydrogel and solutions.

Additionally, natural herbal oils, vitamins, odorizing and coloring agents can be added into the carrier.
The other substance forming the product is the active ingredient. This active ingredient is the exosome isolated from the medium wherein human newborn foreskin stem cells are reproduced from the stem cell isolated from 0 to 5 month old human newborn foreskin stem cells.
The said product contains 0.5-5 μg active ingredient/100 ml carrier by mass (w/w).
The obtained end product of the present invention can be in the form of a cream, ointment, lotion, gel, spray, solution or foam.

### Experiment Results

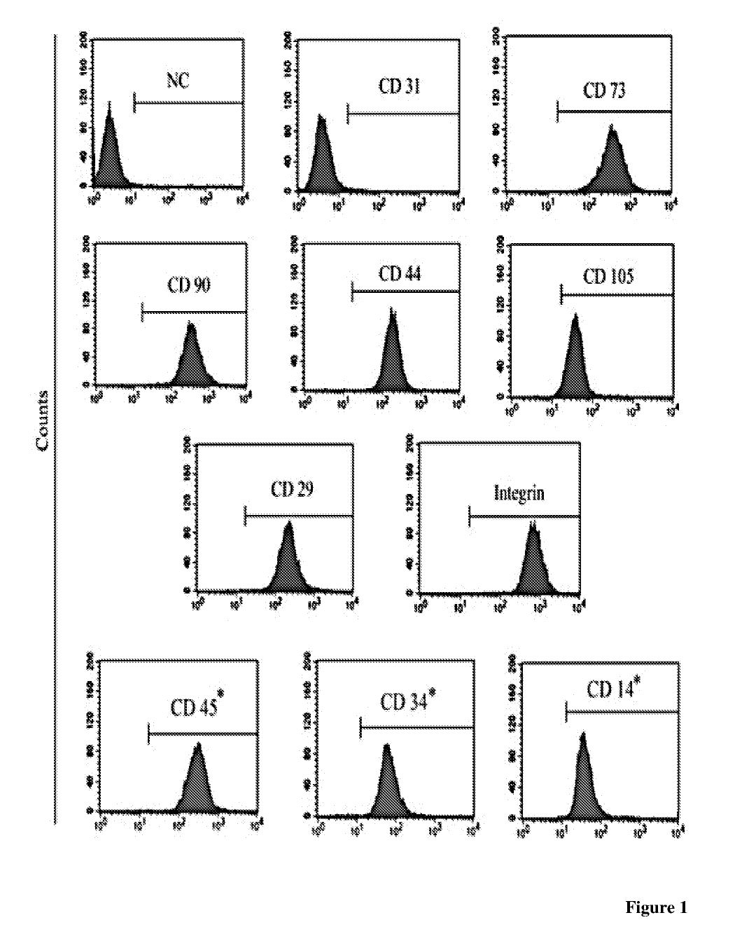

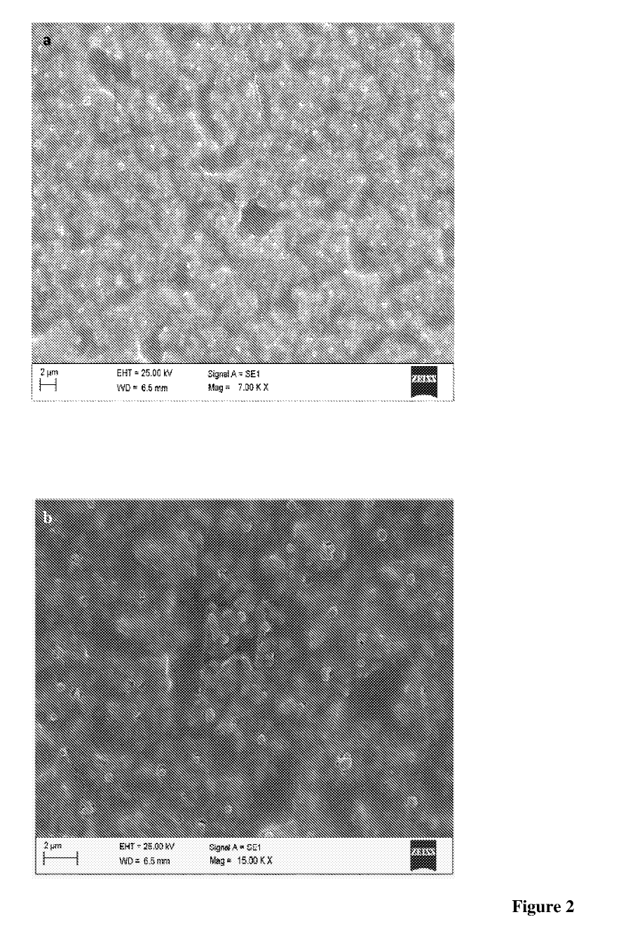

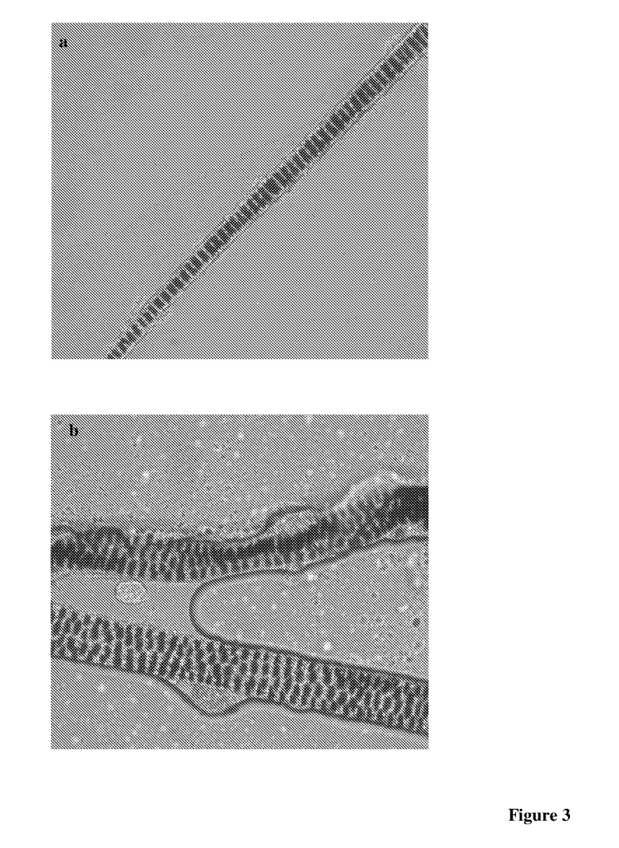

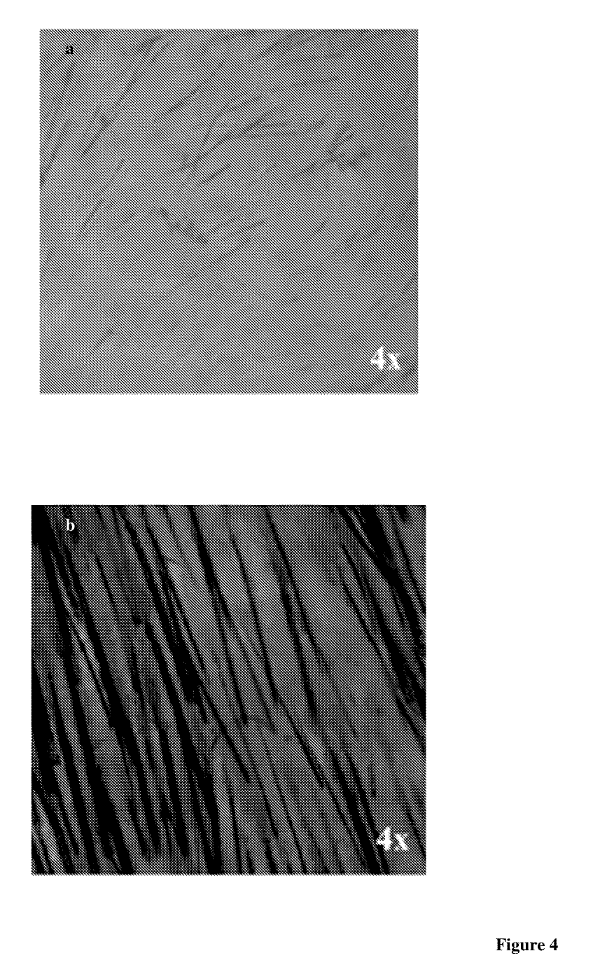

As a result of the experimental studies conducted, analyses of the surface markers as a result of flow cytometry are shown in FIG. 1. The fact that both hematopoietic and mesenchymal markers are positive demonstrates that these stem cells have much better regeneration and differentiation potentials than the other stem cells (FIG. 1).

Images of the isolated exosomes were taken by means of ZEISS SEM Electron Microscope. In the obtained images, the exosomes were found to have a diameter of 20-60 nm. In fact, diameters of exosomes vary between 40 and 120 nm. The fact that the exosomes of human newborn foreskin stem cells are smaller shows that when it is applied, interaction with the plasma membrane will be better and it will increase stimulation of the hair stem cells (FIG. 2).

The keratinocyte vesicle in the hair of the mouse on which the product of the invention is not applied is positioned as a single row (FIG. 3-a). The keratinocyte vesicle in the hair of the mouse on which the product of the invention is applied is positioned as four rows. This shows that the newly growing hair is four times stronger and thicker than the normal hair (FIG. 3-b).

Exosomes obtained from newborn foreskin stem cells can be used as additives in various cell regeneration products. Furthermore, it can be used in enhancing differentiation abilities of different cell sources, preparing cell medium and enhancing cell viability. For example, it can be used as an active ingredient in skin regeneration and wound healing products.

### Application of the Invention

This product can be administered topically directly on the skin or via macro needle and micro needle methods. Furthermore, the product can also be administered subcutaneously.
The product can be produced in the form of a hair tonic, hair conditioner, hair lotion, hair spray, hair aerosol.
The product of the invention can be used against problems of intense hair loss, baldness, androgenic alopecia, tinea capitis and early hair greying.
This product is applied to the scalp twice a day. In each application, 3 fingertips of the product are applied to the bald areas.
The product of the invention should be stored at +4° C. Furthermore, one should perform the application when her/his body temperature is normal.

1. A component, comprising a carrier and an active ingredient, wherein the component prevents hair loss and enables newly growing hair to be natural and healthy.

2. The component according to claim 1, wherein the carrier comprises one or more of natural and chemical oils, synthetic and semisynthetic polymers, biopolymers, hydrogel and solutions.

3. The component according to claim 2, wherein the carrier comprises one or more of natural herbal oils, vitamins, odorizing and coloring agents.

4. The component according to claim 3, wherein the active ingredient comprises exosome isolated from a medium where 0-5 month old human newborn foreskin stem cells are grown.

5. A product comprising 0.5-5 μg active ingredient and 100 ml carrier by mass (w/w).

6. The product according to claim 5, wherein the product is in the form of a cream, ointment, lotion, gel, spray, solution or foam.

7. The product according to claim 5, wherein the product is produced in the form of a hair tonic, hair conditioner, hair lotion, hair spray, or hair aerosol.

8. The product according to claim 5, wherein 3 fingertips of the product are applied to the skin twice a day.

9. The product according to claim 5, wherein the product can be stored at a temperature of +4° C.

10. The product according to claim 5, wherein the product can be administered subcutaneously, topically on the skin or via macro needle and micro needle method.

11. The product according to claim 5, wherein the product can be used for treatment of problems of intense hair loss, baldness, androgenic alopecia, tinea capitis and early hair greying.

12. The product according to claim 5, wherein the product can be used as active ingredient in skin regeneration and wound healing.
